(19) United States
(12) Patent Application Publication
(10) Pub. No.: US 2026/0262756 A1
(43) Pub. Date: Sep. 10, 2026

(54) SOCK FOR CONTROLLED RELEASE OF SUBSTANCE

(71) Applicant: Core Pacific, Inc., Houston, TX (US)

(72) Inventor: Patrick Gwen, Houston, TX (US)

(21) Appl. No.: 19/560,675

(22) Filed: Mar. 9, 2026

Related U.S. Application Data
(60) Provisional application No. 63/768,451, filed on Mar. 7, 2025.

(51) Int. Cl. A41B 11/00; A61M 35/00

(52) U.S. Cl. CPC A41B 11/00; A61M 35/10; A41B 2400/32; A41B 2400/52

(57) ABSTRACT

A sock configured for the controlled release of a substance, such as one or more materials beneficial for a spa treatment, is described herein. In some embodiments, the sock comprises a first compartment configured to receive a foot of a wearer and a second compartment configured to contain the substance. A substantially impermeable barrier may be disposed between the first compartment and the second compartment, the barrier being configured to be ruptured upon insertion of the foot into the sock, thereby allowing the substance to be released into the inner cavity of the first compartment. The substance may comprise, for example, lotions, oils, paraffin, exfoliants, nutrients, or other cosmetic or therapeutic materials configured to contact the foot of the wearer.

### CROSS-REFERENCE TO RELATED APPLICATIONS

This application is a non-provisional and claims benefit of U.S. Provisional Application No. 63/768,451 filed Mar. 7, 2025, the specification of which is incorporated herein in its entirety by reference.

### FIELD OF THE INVENTION

The present invention features a sock configured for the controlled release of a substance, such as one or more materials beneficial for a spa treatment.

### BACKGROUND OF THE INVENTION

The present invention relates to wearable spa or treatment articles, and more particularly to a sock configured for the controlled release of one or more substances beneficial for spa or therapeutic treatments. Conventional spa socks often fail to retain substances in specific regions of the sock and may require bulky packaging. There is a need for a sock that maintains substances in separate compartments until use, provides controlled release upon insertion of the wearer's foot, and allows for compact and efficient packaging. The present invention addresses these needs by providing a sock comprising a first compartment comprising an opening configured to receive a foot of a wearer, a second compartment for containing one or more substances, and one or more barriers configured to control the release of the substances during use.

### BRIEF SUMMARY OF THE INVENTION

It is an objective of the present invention to provide a sock that allows for the controlled release of a substance, such as one or more materials beneficial for a spa treatment, as specified in the independent claims. Embodiments of the invention are given in the dependent claims. Embodiments of the present invention can be freely combined with each other if they are not mutually exclusive.

The present invention features a sock configured for the controlled release of a substance, such as one or more materials beneficial for a spa treatment. In some embodiments, the sock comprises a first compartment configured to receive a foot of a wearer and a second compartment configured to contain the substance. A substantially impermeable barrier may be disposed between the first compartment and the second compartment, the barrier being configured to be ruptured upon insertion of the foot into the sock, thereby allowing the substance to be released into the inner cavity of the first compartment. The substance may comprise, for example, lotions, oils, paraffin, exfoliants, nutrients, or other cosmetic or therapeutic materials configured to contact the foot of the wearer.

In some embodiments, the present invention features a sock configured to retain a substance therein. In some embodiments, the sock comprises a first compartment comprising an opening and an inner cavity, the opening being configured to receive a foot of a wearer, a second compartment comprising an inner cavity containing the substance, and a substantially impermeable barrier (e.g., at least one substantially impermeable barrier) disposed between the first compartment and the second compartment. In some embodiments, the substantially impermeable barrier is configured such that insertion of the foot of the wearer through the opening causes the foot to contact and pass through a portion of the barrier, thereby rupturing at least the contacted portion of the barrier and releasing the substance from the inner cavity of the second compartment into the inner cavity of the first compartment.

One of the unique and inventive technical features of the present invention is the division of the sock into a first compartment and a second compartment by a barrier. Without wishing to limit the invention to any theory or mechanism, it is believed that the technical feature of the present invention advantageously allows substances to be retained within the second compartment, for example, at the tip of the sock, until use. This compartmentalized design also enables a more compact packaging of the sock. None of the presently known prior references or works has the unique inventive technical feature of the present invention.

Any feature or combination of features described herein are included within the scope of the present invention, provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A-1C shows one embodiment of the sock of the present invention. FIGS. 1B and 1C show front views of the sock with a barrier positioned vertically (FIG. 1B) or horizontally (FIG. 1C). In FIG. 1B, the barrier is shown in a closed configuration (left) and an opened configuration (right).

FIG. 2 shows one embodiment of the sock of the present invention in use.

### DETAILED DESCRIPTION OF THE INVENTION

Following is a list of elements corresponding to a particular element referred to herein:
100 Sock
110 First Compartment
115 Opening
120 Second Compartment
130 Barrier

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

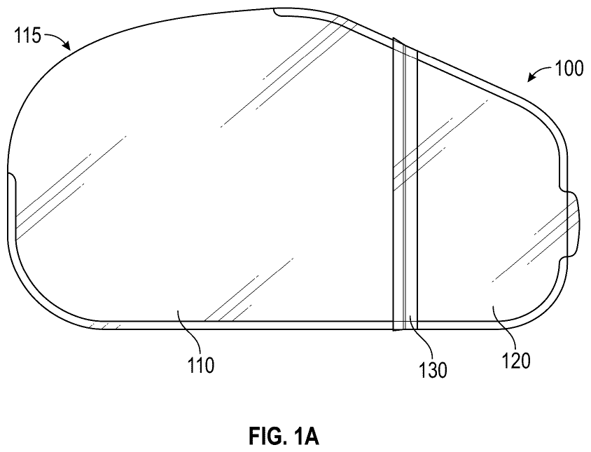

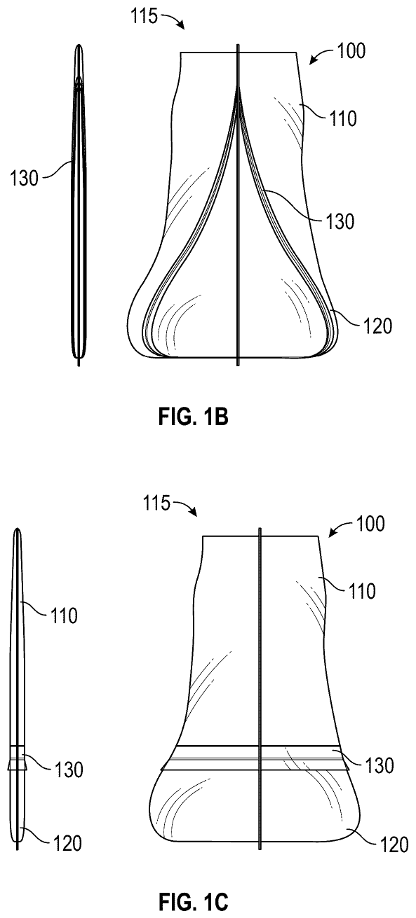

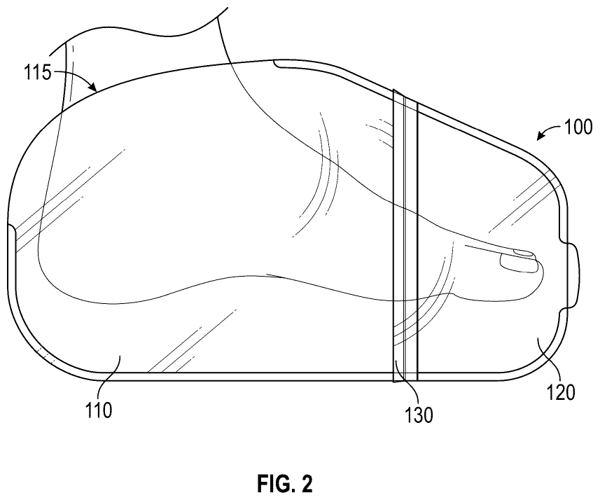

Referring now to FIGS. 1A-2, the present invention features a sock configured for the controlled release of a substance, such as one or more materials beneficial for a spa treatment.

Referring to FIG. 1A, the present invention may feature a sock (100) configured to retain a substance therein. In some embodiments, the sock (100) comprises a first compartment (110) comprising an opening (115) configured to receive a foot of a wearer, a second compartment (120) containing a substance therein, and a barrier (130; e.g., a substantially impermeable barrier) disposed between the first compartment (110) and the second compartment (120). In other embodiments, the sock (100) comprises a first compartment (110) comprising an opening configured to receive a foot of a wearer, a second compartment (120) containing a substance therein, and at least one barrier (130; e.g., a substantially impermeable barrier) disposed between the first compartment (110) and the second compartment (120). In some embodiments, the barrier (130) is configured such that insertion of the foot of the wearer through the opening (115) causes the foot to contact and pass through a portion of the barrier (130), thereby rupturing at least the contacted portion of the barrier (130) and releasing the substance from the second compartment (120) into the first compartment (110).

In some embodiments, the barrier (130; e.g., a substantially impermeable barrier) forms an airtight seal between the first compartment (110) and the second compartment (120) prior to rupture by the foot. In other embodiments, the barrier (130; e.g., a substantially impermeable barrier) forms a watertight seal between the first compartment (110) and the second compartment (120) prior to rupture by the foot. In further embodiments, the barrier (130; e.g., a substantially impermeable barrier) forms an airtight and watertight seal between the first compartment (110) and the second compartment (120) prior to rupture by the foot.

In some embodiments, the sock (100) comprises a first compartment (110) comprising an inner cavity and an opening configured to receive a foot of a wearer, a second compartment (120) comprising an inner cavity having a substance therein, and a barrier (130; e.g., a substantially impermeable barrier) disposed between the first compartment (110) and the second compartment (120), i.e., a barrier (130) disposed between the inner cavity of the first compartment (110) and the inner cavity of the second compartment (120). In some embodiments, the barrier (130; e.g., a substantially impermeable barrier) is configured such that insertion of the foot of the wearer through the opening (115) causes the foot to contact and pass through a portion of the barrier (130), thereby rupturing at least the contacted portion of the barrier (130) and releasing the substance from the inner cavity of the second compartment (120) into the inner cavity of the first compartment (110).

In some embodiments, the barrier (130; e.g., a substantially impermeable barrier) forms an airtight seal between the inner cavity of the first compartment (110) and the inner cavity of the second compartment (120) prior to rupture by the foot. In other embodiments, the barrier (130; e.g., a substantially impermeable barrier) forms a watertight seal between the inner cavity of the first compartment (110) and the inner cavity of the second compartment (120) prior to rupture by the foot. In further embodiments, the barrier (130; e.g., a substantially impermeable barrier) forms an airtight and watertight seal between the inner cavity of the first compartment (110) and the inner cavity of the second compartment (120) prior to rupture by the foot.

In some embodiments, the aforementioned sock (100) may further comprise a second barrier (e.g., a second substantially impermeable barrier). In some embodiments, the second barrier divides the second compartment (120), or the inner cavity of the second compartment (120), into a first portion and a second portion. In some embodiments, the sock (100) may further comprise a second barrier configured to divide the second compartment (120), or the inner cavity thereof, into a first portion and a second portion, each portion configured to contain a different substance.

In some embodiments, the second barrier (e.g., the second substantially impermeable barrier) is configured such that insertion of the foot of the wearer through the first barrier (130) causes the foot to contact and pass through a portion of the second barrier, thereby rupturing at least the contacted portion of the second barrier and releasing the substance from the second portion into the first portion. Without wishing to limit the present invention to any theory or mechanism, it is believed that the presence of one or more additional barriers (e.g., the second barrier) within the second compartment, or within the inner cavity of the second compartment (120), allows substances that are preferably maintained separately to remain separated prior to use of the sock. Upon insertion of the foot into the sock, rupture of the barriers allows the substances contained in the first portion and the second portion to mix.

In some embodiments, the barrier (130; e.g., a substantially impermeable barrier) is configured such that insertion of the foot of the wearer through the opening (115) causes the foot to contact and pass through the entire barrier (130).

In some embodiments, the barrier (130; e.g., a substantially impermeable barrier) comprises an interlocking mechanism. In some embodiments, the interlocking mechanism comprises interlocking plastic ridges. In some embodiments, the barrier (130; e.g., a substantially impermeable barrier) comprises perforations configured to facilitate rupture of the barrier.

In some embodiments, the barrier (130; e.g., a substantially impermeable barrier) is disposed vertically between the first compartment (110) and the second compartment (120), e.g., see FIG. 1B. In such embodiments, the barrier (130; e.g., a substantially impermeable barrier) is perpendicular to the foot of the wearer. In other embodiments, the barrier (130; e.g., a substantially impermeable barrier) is disposed horizontally between the first compartment (110) and the second compartment (120), e.g., see FIG. 1C. In such embodiments, the barrier (130; e.g., a substantially impermeable barrier) is perpendicular to the foot of the wearer. In some embodiments in which the sock (100) comprises one or more additional barriers (e.g., a second barrier), the additional barriers may be disposed within the second compartment (120) in a vertical or horizontal orientation. However, the present invention is not limited to barriers arranged in a vertical or horizontal orientation, and the barriers may be disposed at any suitable angle within the sock.

In some embodiments, the first compartment (110) is larger than the second compartment (120).

In some embodiments, the opening (115) is initially sealed prior to use. In certain embodiments, the opening (115) is sealed by a removable seal configured to be opened prior to insertion of a foot. In some embodiments, the seal comprises a perforated region configured to be torn or cut to form the opening (115).

In some embodiments, the socks (100) described herein further comprise one or more adhesives disposed on an outer surface surrounding the opening (115). In such embodiments, the sock (100) may further comprise a removable release liner covering the one or more adhesives disposed around the opening (115). In some embodiments, one or more adhesives are disposed at or near the level of the ankle of the wearer and are configured to secure the opening around the ankle of the wearer sufficiently to reduce or prevent leakage of the substance from the sock.

In some embodiments, the substance comprises one or more materials beneficial for a spa treatment. In some embodiments, the substance comprises one or more materials beneficial for a spa treatment, including, but not limited to, lotions, paraffin, oils, gels, nutrients, vitamins, moisturizers, exfoliants, or other cosmetic, therapeutic, or skin-conditioning agents. In some embodiments, the substance may be a liquid, semi-solid, or solid. In certain embodiments, the substance can include one or more components that are maintained separately prior to use, for example, to prevent premature mixing or degradation.

In some embodiments, the socks (100) described herein are disposable. In some embodiments, the socks (100) described herein are made of an impermeable material.

Embodiments:

The following embodiments are intended to be illustrative only and not to be limiting in any way.
Embodiment 1: A sock (100) configured to retain a substance therein, the sock (100) comprising: a) a first compartment (110) comprising an opening (115) and an inner cavity, the opening (115) being configured to receive a foot of a wearer; b) a second compartment (120) comprising an inner cavity containing the substance; and c) a substantially impermeable barrier (130; e.g., at least one substantially impermeable barrier) disposed between the first compartment (110) and the second compartment (120), wherein the substantially impermeable barrier (130) is configured such that insertion of the foot of the wearer through the opening (115) causes the foot to contact and pass through a portion of the barrier (130), thereby rupturing at least the contacted portion of the barrier (130) and releasing the substance from the inner cavity of the second compartment (120) into the inner cavity of the first compartment (110).
Embodiment 2: The sock of embodiment 1, wherein the substantially impermeable barrier (130) is configured such that insertion of the foot of the wearer through the opening (115) causes the foot to contact and pass through the entire barrier (130).
Embodiment 3: The sock of embodiment 1 or embodiment 2, wherein the substantially impermeable barrier (130) forms an airtight seal between the first compartment (110) and the second compartment (120) prior to rupture by the foot. Embodiment 4: The sock of any one of embodiments 1-3, wherein the substantially impermeable barrier (130) forms a watertight seal between the first compartment (110) and the second compartment (120) prior to rupture by the foot.
Embodiment 5: The sock of any one of embodiments 1-4, wherein the substantially impermeable barrier (130) comprises an interlocking mechanism. Embodiment 6: The sock of embodiment 6, wherein the interlocking mechanism comprises interlocking plastic ridges.
Embodiment 7: The sock of any one of embodiments 1-6, wherein the substantially impermeable barrier (130) is disposed vertically between the first compartment (110) and the second compartment (120). Embodiment 8: The sock of embodiment 7, wherein the substantially impermeable barrier (130) is perpendicular to the foot of the wearer. Embodiment 9: The sock of any one of embodiments 1-6, wherein the substantially impermeable barrier (130) is disposed horizontally between the first compartment (110) and the second compartment (120). Embodiment 10: The sock of embodiment 9, wherein the substantially impermeable barrier (130) is parallel to the foot of the wearer.
Embodiment 11: The sock of any one of embodiments 1-10, wherein the first compartment (110) is larger than the second compartment (120).
Embodiment 12: The sock of any one of embodiments 1-11, further comprising a second substantially impermeable barrier, wherein the second substantially impermeable barrier divides the second compartment (120) into a first portion and a second portion. Embodiment 13: The sock of embodiment 12, wherein the second substantially impermeable barrier forms an airtight seal between the first portion and the second portion prior to rupture by the foot. Embodiment 14: The sock of embodiment 12 or embodiment 13, wherein the second substantially impermeable barrier forms a watertight seal between the first portion and the second portion prior to rupture by the foot.
Embodiment 15: The sock of any one of embodiments 12-14, wherein the second substantially impermeable barrier comprises an interlocking mechanism. Embodiment 16: The sock of embodiment 15, wherein the interlocking mechanism comprises interlocking plastic ridges.
Embodiment 17: The sock of any one of embodiments 12-16, wherein the second substantially impermeable barrier is disposed vertically between the first portion and the second portion of the second compartment (120). Embodiment 18: The sock of embodiment 17, wherein the second substantially impermeable barrier is perpendicular to the foot of the wearer. Embodiment 19: The sock of any one of embodiments 12-16, wherein the second substantially impermeable barrier is disposed horizontally between the first portion and the second portion of the second compartment (120). Embodiment 20: The sock of embodiment 19, wherein the substantially impermeable barrier (130) is parallel to the foot of the wearer.
Embodiment 21: The sock of any one of embodiments 1-20, wherein the opening (115) is initially sealed prior to use. Embodiment 22: The sock of embodiment 21, wherein the opening (115) is sealed by a removable seal configured to be opened prior to insertion of a foot. Embodiment 23: The sock of embodiment 21, wherein the seal comprises a perforated region configured to be torn or cut to form the opening (115). Embodiment 24: The sock of any one of embodiments 1-21, further comprising one or more adhesives disposed on an outer surface surrounding the opening (115). Embodiment 25: The sock of embodiment 24, further comprising a removable release liner covering the one or more adhesives disposed around the opening (115).
Embodiment 26: The sock of any one of embodiments 1-22, wherein the substance comprises one or more materials beneficial for a spa treatment. Embodiment 27: The sock of embodiment 23, wherein the material beneficial for a spa treatment comprises lotions, paraffin, oils, gels, nutrients, vitamins, moisturizers, and exfoliants. Embodiment 28: The sock of any one of embodiments 1-27, wherein the substance is liquid. Embodiment 29: The sock of any one of embodiments 1-27, wherein the substance is semi-solid. Embodiment 30: The sock of any one of embodiments 1-27, wherein the substance is solid. Embodiment 31: The sock of any one of embodiments 1-30, wherein the substance includes one or more components maintained separately prior to use.
Embodiment 32: The sock of any one of embodiments 1-31, wherein the sock is disposable. Embodiment 33: The sock of any one of embodiments 1-32, wherein the sock is made from an impermeable material.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Reference numbers recited herein, in the drawings, and in the claims are solely for ease of examination of this patent application and are exemplary. The reference numbers are not intended in any way to limit the scope of the claims to the particular features having the corresponding reference numbers in the drawings.

What is claimed is:

1. A sock (100) configured to retain a substance therein, the sock (100) comprising:
a) a first compartment (110) comprising an opening (115) and an inner cavity, the opening (115) being configured to receive a foot of a wearer;
b) a second compartment (120) comprising an inner cavity containing the substance; and
c) a substantially impermeable barrier (130) disposed between the first compartment (110) and the second compartment (120),
wherein the substantially impermeable barrier (130) is configured such that insertion of the foot of the wearer through the opening (115) causes the foot to contact and pass through a portion of the barrier (130), thereby rupturing at least the contacted portion of the barrier (130) and releasing the substance from the inner cavity of the second compartment (120) into the inner cavity of the first compartment (110).

2. The sock (100) of claim 1, wherein the substantially impermeable barrier (130) is configured such that insertion of the foot of the wearer through the opening (115) causes the foot to contact and pass through the entire barrier (130).

3. The sock (100) of claim 1, wherein the substantially impermeable barrier (130) forms an airtight seal and/or a watertight seal between the first compartment (110) and the second compartment (120) prior to rupture by the foot.

4. The sock (100) of claim 1, wherein the substantially impermeable barrier (130) comprises an interlocking mechanism.

5. The sock (100) of claim 4, wherein the interlocking mechanism comprises interlocking plastic ridges.

6. The sock (100) of claim 1, wherein the substantially impermeable barrier (130) is disposed vertically between the first compartment (110) and the second compartment (120).

7. The sock (100) of claim 6, wherein the substantially impermeable barrier (130) is perpendicular to the foot of the wearer.

8. The sock (100) of claim 1, wherein the substantially impermeable barrier (130) is disposed horizontally between the first compartment (110) and the second compartment (120).

9. The sock of claim 8, wherein the substantially impermeable barrier (130) is parallel to the foot of the wearer.

10. The sock (100) of claim 1, wherein the first compartment (110) is larger than the second compartment (120).

11. The sock (100) of claim 1, further comprising a second substantially impermeable barrier, wherein the second substantially impermeable barrier divides the second compartment (120) into a first portion and a second portion.

12. The sock (100) of claim 11, wherein the second substantially impermeable barrier forms an airtight and/or a watertight seal between the first portion and the second portion prior to rupture by the foot.

13. The sock (100) of claim 11, wherein the second substantially impermeable barrier comprises an interlocking mechanism, wherein the interlocking mechanism comprises interlocking plastic ridges.

14. The sock (100) of claim 11, wherein the second substantially impermeable barrier is disposed vertically between the first portion and the second portion of the second compartment (120).

15. The sock (100) of claim 11, wherein the second substantially impermeable barrier is disposed horizontally between the first portion and the second portion of the second compartment (120).

16. The sock (100) of claim 1, wherein the opening (115) is initially sealed prior to use, wherein the opening (115) is sealed by a removable seal configured to be opened prior to insertion of a foot.

17. The sock (100) of claim 1, further comprising one or more adhesives disposed on an outer surface surrounding the opening (115).

18. The sock (100) of claim 17, further comprising a removable release liner covering the one or more adhesives disposed around the opening (115).

19. The sock (100) of claim 1, wherein the substance comprises one or more materials beneficial for a spa treatment, selected from the group consisting of lotions, paraffin, oils, gels, nutrients, vitamins, moisturizers, and exfoliants.

20. The sock (100) of claim 1, wherein the sock is disposable.
